(19) United States
(12) Patent Application Publication
(10) Pub. No.: US 2011/0150891 A1
(43) Pub. Date: Jun. 23, 2011

(54) Methods of Treating Interstitial Cystitis

(76) Inventor: Philip Bosch, Fallbrook, CA (US)

(21) Appl. No.: 12/969,923

(22) Filed: Dec. 16, 2010

Related U.S. Application Data
(60) Provisional application No. 61/287,134, filed on Dec. 16, 2009.

(51) Int. Cl. A61K 39/395; A61P 13/10; A61P 29/00

(52) U.S. Cl. 424/142.1

(57) ABSTRACT

The present invention relates to the use of adalimumab (Humira™), for the treatment of a pain and/or a lower urinary tract symptom(s) (LUTS) associated with interstitial cystitis and/or painful bladder syndrome and/or bladder pain syndrome.

### RELATED APPLICATIONS

This application claims the benefit of U.S. Ser. No. 61/287134, filed Dec. 16, 2009 the contents of which is incorporated herein by reference in its entirety.

### FIELD OF THE INVENTION

The present invention relates to the treatment of the signs and symptoms associated with interstitial cystitis with a TNF-a antagonist such as adalimumab (i.e., Humira®)

### BACKGROUND OF THE INVENTION

Interstitial Cystitis (IC) is a debilitating bladder disease of uncertain etiology. It afflicts as many as one million patients in the United States with females comprising 90%¹ of those patients. Symptoms include bladder, pelvic, and or perineal pain, urinary frequency, urgency, and nocturia. These symptoms result in such severe morbidity that patients with IC score worse on quality of life tests than patients on terminal dialysis².

The diagnosis of IC has always been a challenge³. There is usually a delay, most commonly years, in the diagnosis of a patient with IC. There is no specific diagnostic test that unequivocally establishes the diagnosis of IC. The patient's symptoms will vary but will include bladder pain on bladder filling, urinary frequency, urgency, nocturia and in women, dysparunia. Questionnaires have been developed to screen patients for IC including the O'Leary-Sant Symptom Index and Problem Index⁴. A voiding diary can be helpful not only in diagnosing the patient but also in evaluating the effectiveness of treatment. The patient will often have normal urine analyses and urine cultures. However, many patients with IC will have microscopic hematuria. Physical exam is normal except for bladder tenderness in both abdominal and bimanual exam. Cystoscopy under anesthesia with bladder hydro distension will show petechia of the bladder wall, which is consistent with IC. It may also show a Hunner's ulcer, which is diagnostic for IC. A biopsy of the bladder demonstrates inflammation with increased mast cells. A potassium sensitivity test demonstrates increased bladder discomfort when a liquid solution of potassium is instilled in the bladder. A thorough evaluation of the patient, to rule out other diseases, will lead to the correct diagnosis of IC.

There is no cure for IC and treatment is limited to symptomatic relief. Patients should avoid certain foods that irritate their bladder⁵. There are only two drugs that are FDA approved for the treatment of IC. In 1978 the FDA approved dimethyl sulfoxide (DMSO) for the treatment of IC⁶. In September of 1996 the FDA approved Elmiron (pentosan polysulfate sodium) for the treatment of IC⁷. These drugs help only 40 to 80% of the patients and those patients only notice a partial improvement^{8,9}. Most IC patients are still symptomatic and are living in discomfort with daily bladder symptoms. There has been no new treatment for IC for many years. Thus a needs exists to provide a novel, effective treatment for a pain and/or a lower urinary tract symptom of interstitial cystitis and/or painful bladder syndrome and/or bladder pain syndrome, without the adverse effects or limited efficacy of currently available therapies.

### SUMMARY OF THE INVENTION

The present invention provides methods of treating or alleviating a symptom of interstitial cystitis in a subject in need thereof by administering a therapeutically effective amount of Humira®. Humira® can be administered by any methods known in the art. Preferably, Humira® is administered subcutaneously. A therapeutically effective amount is nay amount that has a clinical benefit, i.e., alleviates at least one symptom of interstitial cystitis. Preferably the therapeutically effective amount is 40 mg.

In some aspects Humira® is administered in an initial loading dose followed by a maintenance dose. Optionally, a second loading does is administered prior to the maintenance dose. In some embodiments the initial loading dose is administered over two consecutive days.

A loading dose is for example 160 mg or 80 mg. A maintenance dose is 40 mg. The maintenance dose is administered bi-weekly or every ten days.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### DETAILED DESCRIPTION OF THE INVENTION

The invention provides improved uses and compositions for treating of a pain and/or a lower urinary tract symptom(s) (LUTS) associated with interstitial cystitis and/or painful bladder syndrome and/or bladder pain syndrome with a TNFα inhibitor, e.g., a human TNFα antibody, or an antigen-binding portion thereof. Preferably, the TNFα inhibitor is Humira®. Compositions and articles of manufacture, including kits, relating to the methods and uses for treating interstitial cystitis are also contemplated as part of the invention.

Interstitial cystitis (IC) is a chronic condition affecting primarily the bladder and is of unknown origin. IC is characterized by symptoms of pain, such as pelvic pain, and lower urinary tract symptom(s) (LUTS), such as increased urinary frequency or urgency (particularly persistent urge). More recently terminology has evolved to include painful bladder syndrome (PBS) (MacDiarmid et al, Rev. Urol., 9(1), 9-1 6 (2007)) or bladder pain syndrome (BPS) (van der Merve et al, European Urology, 53, 60-67 (2008)), along with IC, that is IC/PBS/BPS to collectively describe this symptom complex.

Pain associated with IC, PBS or BPS comprises lower abdominal (pelvic) pain, bladder pain, suprapubic pain, vaginal pain, pain in the penis, testicles, scrotum or perineum, urethral pain, dyspareneuria, or pain, pressure or discomfort that may increase as the bladder fills.

Lower urinary tract symptoms comprise three groups of urinary symptoms, which may be defined as storage (irritative), voiding (obstructive) and post-micturition symptoms.

Storage symptoms comprise urgency, frequency, nocturia, urgency incontinence and stress incontinence. Voiding symptoms comprise hesitancy, poor flow, intermittency, straining and dysuria. Post-micturition symptoms comprise terminal dribbling, post-void dribbling and a sense of incomplete emptying. The term ‘urgency’ is defined by the International Continence Society as the complaint of a sudden compelling desire to pass urine which is difficult to defer. This may be associated with a concern or fear of incontinence, a concern or fear of worsening pain, pressure or discomfort, or a concern or fear of onset or worsening of another unpleasant symptom related to the lower urinary tract. In some patients with interstitial cystitis/painful bladder syndrome/bladder pain syndrome, this sensation of urgency may be accompanied by an increasing feeling of malaise and/or nausea.

The etiology and pathophysiology of IC has not been definitively established. Numerous theories have been proposed. These theories include autoimmunity, disruption of the glycosaminoglycan (GAG) protective layer of the bladder mucosa, and sensory nerves releasing inflammatory neuropeptides¹⁰. The offending etiological agent ultimately provokes bladder urothelial inflammation, resulting in associated irritative symptoms^{11.}

Clinical and experimental models of IC pathogenesis involve the inflammatory mediators released by mast cells. Excessive mast cells in the bladder muscularis are seen in bladder biopsies of patients with IC^{12,13} and experimental mice studies for IC¹⁴. Mast cell numbers are often increased in several bladder syndromes and this mast cell influx has been observed in bladder cancer, interstitial cystitis and chronic cystitis^{15,16}. Mast cell activation has been demonstrated in interstitial cystitis¹⁷. The possibility that mast cells are important in bladder pathogenesis is also consistent with their demonstrated importance in inflammatory diseases, such as asthma, irritable bowel disease, arthritis, and atopic dermatitis, and Crohn's disease'^{18,19,20}.

Mast cell inflammatory response may be mediated by tumor necrosis factor (TNF)^{21,22} Intravesical suplatast tosilate inhibits the release of tumor necrosis factor by effecting mast cell secretion in an experimental model to inhibit bladder inflammation²³. Intravesical nanocrystalline silver inhibits the release of tumor necrosis factor by effecting mast cell secretion in an experimental model to inhibit bladder inflammation and may be useful in interstitial cystitis²⁴. There is a report in an Interstitial Cystitis Support Group forum of a 63 year old female with a 37 year history of IC who received relief from Remicade (inflaximab)²⁵. Remicade® is also a TNF blocker and it was used as an infusion every two months. There was a review article on IC where the importance of mast cell activation with the release of TNF is discussed²⁶. They state that Remicade® and Embrel® block TNF but have never been used for the treatment of IC. Inhibiting the activation of mast cell response and decreasing the effect of tumor necrosis factor may be useful in treating interstitial cystitis.

Humira is a medicine that is a TNF blocker. Humira® has been shown to be beneficial in other inflammatory diseases such as rheumatoid arthritis, polyarticular idiopathic arthritis, psoriatic arthritis, and Crohn's disease. Humira® should be beneficial in the treatment of IC.

### Definitions

The term “human TNFα” (abbreviated herein as hTNFα, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNFα is described further in, for example, Pennica, D., et al. (1984) Nature 312:724-729; Davis, J. M., et al. (1987) Biochemistry 26:1322-1326; and Jones, E. Y., et al. (1989) Nature 338:225-228. The term human TNFα is intended to include recombinant human TNFα (rhTNFα), which can be prepared by standard recombinant expression methods or purchased commercially (R & D Systems, Catalog No. 210-TA, Minneapolis, Minn.). TNFα is also referred to as TNF.

The term “TNFα inhibitor” includes agents which interfere with TNFα activity. The term also includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356. In one embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein. In another embodiment, the TNFα inhibitor is a TNF fusion protein, e.g., etanercept (Enbrel®, Amgen; described in WO 91/03553 and WO 09/406,476, incorporated by reference herein). In another embodiment, the TNFα inhibitor is a recombinant TNF binding protein (r-TBP-I) (Serono).

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The antibodies of the invention are described in further detail in U.S. Pat. Nos. 6,090,382; 6,258,562; and 6,509,015, each of which is incorporated herein by reference in its entirety.

The term “antigen-binding portion” or “antigen-binding fragment” of an antibody (or simply “antibody portion”), as used herein, refers to one or more fragments of an antibody that retain the ability to specifically bind to an antigen (e.g., hTNFα). It has been shown that the antigen-binding function of an antibody can be performed by fragments of a full-length antibody. Binding fragments include Fab, Fab′, F(ab′)₂, Fabc, Fv, single chains, and single-chain antibodies. Examples of binding fragments encompassed within the term “antigen-binding portion” of an antibody include (i) a Fab fragment, a monovalent fragment consisting of the VL, VH, CL and CH1 domains; (ii) a F(ab')₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). The antibody portions of the invention are described in further detail in U.S. Pat. Nos. 6,090,382, 6,258,562, 6,509,015, each of which is incorporated herein by reference in its entirety.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab')₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Chimeric antibodies” refers to antibodies wherein one portion of each of the amino acid sequences of heavy and light chains is homologous to corresponding sequences in antibodies derived from a particular species or belonging to a particular class, while the remaining segment of the chains is homologous to corresponding sequences from another species. In one embodiment, the invention features a chimeric antibody or antigen-binding fragment, in which the variable regions of both light and heavy chains mimics the variable regions of antibodies derived from one species of mammals, while the constant portions are homologous to the sequences in antibodies derived from another species. In a preferred embodiment of the invention, chimeric antibodies are made by grafting CDRs from a mouse antibody onto the framework regions of a human antibody.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition to the grafting of the CDRs, humanized antibodies typically undergo further alterations in order to improve affinity and/or immunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Such chimeric, humanized, human, and dual specific antibodies can be produced by recombinant DNA techniques known in the art, for example using methods described in PCT International Application No. PCT/US 86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Nat. Acad. Sci. USA 84:3439-3443; Liu et al. (1987) J. Immunol. 139:3521-3526; Sun et al. (1987) Proc. Nat. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539.

An “isolated antibody”, as used herein, is intended to refer to an antibody that is substantially free of other antibodies having different antigenic specificities (e.g., an isolated antibody that specifically binds hTNFα is substantially free of antibodies that specifically bind antigens other than hTNFα). An isolated antibody that specifically binds hTNFα may, however, have cross-reactivity to other antigens, such as TNFα molecules from other species. Moreover, an isolated antibody may be substantially free of other cellular material and/or chemicals.

A “neutralizing antibody”, as used herein (or an “antibody that neutralized hTNFα activity”), is intended to refer to an antibody whose binding to hTNFα results in inhibition of the biological activity of hTNFα. This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art (see U.S. Pat. No. 6,090,382). Preferably, the ability of an antibody to neutralize hTNFα activity is assessed by inhibition of hTNFα-induced cytotoxicity of L929 cells. As an additional or alternative parameter of hTNFα activity, the ability of an antibody to inhibit hTNFα-induced expression of ELAM-1 on HUVEC, as a measure of hTNFα-induced cellular activation, can be assessed.

The term “surface plasmon resonance”, as used herein, refers to an optical phenomenon that allows for the analysis of real-time biospecific interactions by detection of alterations in protein concentrations within a biosensor matrix, for example using the BlAcore system (Pharmacia Biosensor AB, Uppsala, Sweden and Piscataway, N.J.). For further descriptions, see Example 1 of U.S. Pat. No. 6,258,562 and Jonsson et al. (1993) Ann. Biol. Clin. 51:19; Jönsson et al. (1991) Biotechniques 11:620-627; Johnsson et al. (1995) J. Mol. Recognit. 8:125; and Johnnson et al. (1991) Anal. Biochem. 198:268.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The term “IC₅₀” as used herein, is intended to refer to the concentration of the inhibitor required to inhibit the biological endpoint of interest, e.g., neutralize cytotoxicity activity.

An “effective amount” of a compound or pharmaceutical composition is an amount sufficient to effect beneficial or desired results including clinical results such as alleviation or reduction in pain sensation. An effective amount can be administered in one or more administrations. For purposes of this invention, an effective amount of drug, compound, or pharmaceutical composition is an amount sufficient to treat, ameliorate, reduce the intensity of and/or prevent a pain or a lower urinary tract symptom associated with interstitial cystitis and/or painful bladder syndrome and/or bladder pain syndrome. In some embodiments, the “effective amount” may reduce pain at rest (resting pain) or mechanically-induced pain (including pain following movement), or both, and it may be administered before, during or after painful stimulus. As is understood in the clinical context, an effective amount of a compound or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “dose,” as used herein, refers to an amount of TNFα inhibitor which is administered to a subject.

The term “dosing”, as used herein, refers to the administration of a substance (e.g., an anti-TNFα antibody) to achieve a therapeutic objective (e.g., treatment of interstitial cystitis).

A “dosing regimen” describes a treatment schedule for a TNFα inhibitor, e.g., a treatment schedule over a prolonged period of time and/or throughout the course of treatment, e.g. administering a first dose of a TNFα inhibitor at week 0 followed by a second dose of a TNFα inhibitor on a biweekly dosing regimen. Alternatively, a first dose of a TNFα inhibitor at week 0 followed by a second dose of a TNFα inhibitor every ten days.

The term “multiple-variable dose” includes different doses of a TNFα inhibitor which are administered to a subject for therapeutic treatment. “Multiple-variable dose regimen” or “multiple-variable dose therapy” describes a treatment schedule which is based on administering different amounts of TNFα inhibitor at various time points throughout the course of treatment. Multiple-variable dose regimens are described in PCT application no. PCT/U.S. 05/12007 and U.S. 20060009385, which is incorporated by reference herein.

The term “maintenance therapy” or “maintenance dosing regime” refers to a treatment schedule for a subject or patient diagnosed with a disorder/disease, e.g., interstitial cystitis, to enable them to maintain their health in a given state, e.g, remission. Generally, the first goal of treatment of interstitial cystitis is to induce remission in the subject in need thereof. The next challenge is to keep the subject in remission. Maintenance doses may be used in a maintenance therapy for maintaining remission in a subject who has achieved remission of a disease or who has reached a state of the disease which is advantageous, e.g. reduction in symptoms. In one embodiment, a maintenance therapy of the invention is used for a subject or patient diagnosed with a disorder/disease, e.g., interstitial cystitis to enable them to maintain their health in a state which is completely free of symptoms associated with the disease. In one embodiment, a maintenance therapy of the invention is used for a subject or patient diagnosed with a disorder/disease, e.g., interstitial cystitis, to enable them to maintain their health in a state which is substantially free of symptoms associated with the disease. In one embodiment, a maintenance therapy of the invention is used for a subject or patient diagnosed with a disorder/disease, e.g., interstitial cystitis, to enable them to maintain their health in a state where there is a significant reduction in symptoms associated with the disease.

The term “induction dose” or “loading dose,” used interchangeably herein, refers to the first dose of TNFα inhibitor which is initially used to induce remission of interstitial cystitis. Often, the loading dose is larger in comparison to the subsequent maintenance or treatment dose.

The induction dose can be a single dose or, alternatively, a set of doses. For example, the induction dose is administered in two doses. In one embodiment, an induction dose is subsequently followed by administration of smaller doses of TNFα inhibitor, e.g., the treatment or maintenance dose. The induction dose is administered during the induction or loading phase of therapy. In one embodiment of the invention, the induction dose is at least twice the given amount of the treatment dose. In one embodiment of the invention, the induction dose is 160 mg. In one embodiment of the invention, the induction dose is 80 mg. In on embodiment a second induction dose is given two weeks after the first induction dose. For example a first induction dose of 160 mg is given at week 0 and a second induction dose of 80 mg is given at week 2. The first induction dose can be given in a single day or can be administered over two consecutive days

The term “treatment phase” or “maintenance phase”, as used herein, refers to a period of treatment comprising administration of a TNFα inhibitor to a subject in order to maintain a desired therapeutic effect, i.e., maintaining remission of interstitial cystitis.

The term “maintenance dose” or “treatment dose” is the amount of TNFα inhibitor taken by a subject to maintain or continue a desired therapeutic effect. A maintenance dose can be a single dose or, alternatively, a set of doses. A maintenance dose is administered during the treatment or maintenance phase of therapy. In one embodiment, amaintenance dose(s) is smaller than the induction dose(s) and can be equal to each other when administered in succession. In one embodiment, the invention provides a maintenance dose of 40 mg of adalimumab administered subcutaneously to a subject who is in remission, every other week, or biweekly. In one embodiment, the maintenance dose is administered every other week beginning at week 1 of treatment. Alternatively, the the maintenance dose is administered every other week beginning at week 2, week 3, or week 4 of treatment. Optionally, the maintenance dose is administered every 10 days after the initial loading dose.

The terms “biweekly dosing regimen”, “biweekly dosing”, and “biweekly administration”, as used herein, refer to the time course of administering a substance (e.g., an anti-TNFα antibody) to a subject to achieve a therapeutic objective, e.g, throughout the course of treatment. The biweekly dosing regimen is not intended to include a weekly dosing regimen. Preferably, the substance is administered every 9-19 days, more preferably, every 11-17 days, even more preferably, every 13-15 days, and most preferably, every 14 days. In one embodiment, the biweekly dosing regimen is initiated in a subject at week 0 of treatment. In another embodiment, a maintenance dose is administered on a biweekly dosing regimen. In one embodiment, both the loading and maintenance doses are administered according to a biweekly dosing regimen. In one embodiment, biweekly dosing includes a dosing regimen wherein doses of a TNFα inhibitor are administered to a subject every other week beginning at week 0, week 1, week 2, week 3, week 4. In one embodiment, biweekly dosing includes a dosing regimen where doses of a TNFα inhibitor are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing methods are also described in US 20030235585, incorporated by reference herein.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an anti-TNFα antibody and another drug. The other drug(s) may be administered concomitant with, prior to, or following the administration of an anti-TNFα antibody.

As used herein, “treatment” embraces curative, palliative, suppressive measures and prophylactic treatment and is an approach for obtaining beneficial or desired clinical results. For purposes of this invention, beneficial or desired clinical results include, but are not limited to, one or more of the following: improvement or alleviation of any aspect of a pain and/or a lower urinary tract symptom associated with interstitial cystitis and/or painful bladder syndrome and/or bladder pain syndrome. For purposes of this invention, beneficial or desired clinical results include, but are not limited to, one or more of the following: lessening severity, alleviation of a pain and/or a lower urinary tract symptom associated with interstitial cystitis and/or painful bladder syndrome and/or bladder pain syndrome, including any aspect of pain (such as shortening duration of pain, reduction of pain sensitivity or sensation).

For example, the term treatment may include administration of a TNFα inhibitor prior to or following the onset of interstitial cystitis and/or painful bladder syndrome and/or bladder pain syndrome thereby preventing or removing signs of the disease or disorder. As another example, administration of a TNFα inhibitor after clinical manifestation of interstitial cystitis and/or painful bladder syndrome and/or bladder pain syndrome to combat the symptoms and/or complications and disorders associated with interstitial cystitis and/or painful bladder syndrome and/or bladder pain syndrome comprises “treatment” of the disease. Further, administration of the agent after onset and after clinical symptoms and/or complications have developed where administration affects clinical parameters of the disease or disorder and perhaps amelioration of the disease, comprises “treatment” of interstitial cystitis and/or painful bladder syndrome and/or bladder pain syndrome. In one embodiment, treatment of interstitial cystitis in a subject comprises inducing and maintaining remission of interstitial cystitis and/or painful bladder syndrome and/or bladder pain syndrome in a subject.

“Reducing incidence” of pain and/or a lower urinary tract symptom associated with interstitial cystitis and/or painful bladder syndrome and/or bladder pain syndrome means any of reducing severity (which can include reducing need for and/or amount of (e.g., exposure to) other drugs and/or therapies generally used for this conditions, including, for example, opiates), duration, and/or frequency (including, for example, delaying or increasing time pain in an individual). As is understood by those skilled in the art, individuals may vary in terms of their response to treatment, and, as such, for example, a “method of reducing incidence of pain and/or a lower urinary tract symptom associated with interstitial cystitis and/or painful bladder syndrome and/or bladder pain syndrome in an individual” reflects administering the compound based on a reasonable expectation that such administration may likely cause such a reduction in incidence in that particular individual.

“Ameliorating” a pain and/or a lower urinary tract symptoms associated with interstitial cystitis and/or painful bladder syndrome and/or bladder pain syndrome means a lessening or improvement of one or more symptoms of pain and/or a lower urinary tract symptom(s) associated with interstitial cystitis and/or painful bladder syndrome and/or bladder pain syndrome as compared to not administering the compound. “Ameliorating” also includes shortening or reduction in duration of a symptom.

“Palliative treatment” of a pain and/or a lower urinary tract symptom associated with interstitial cystitis and/or painful bladder syndrome and/or bladder pain syndrome means lessening the extent of one or more undesirable symptoms in an individual or population of individuals treated with the compound.

As used therein, “delaying” the development of pain means to defer, hinder, slow, retard, stabilize, and/or postpone progression of pain and/or a lower urinary tract symptom associated with interstitial cystitis and/or painful bladder syndrome and/or bladder pain syndrome. This delay can be of varying lengths of time, depending on the history of the disease and/or individuals being treated. As is evident to one skilled in the art, a sufficient or significant delay can, in effect, encompass prevention, in that the individual does not develop pain. A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

“Pain” as used herein refers to pain of any etiology, including acute and chronic pain, and any pain with an inflammatory component. As used herein, “pain” includes nociception and the sensation of pain, and pain can be assessed objectively and subjectively, using pain scores and other methods well-known in the art. The pain can be primary or secondary pain, as is well-known in the art.

“Pain associated with interstitial cystitis and/or painful bladder syndrome and/or bladder pain syndrome” as used herein refers primarily to lower abdominal (pelvic) pain, bladder pain, suprapubic pain, vaginal pain, pain in the penis, testicles, scrotum or perineum, urethral pain, dyspareneuria or pain, pressure or discomfort that may increase as the bladder fills.

“Lower urinary tract symptoms associated with interstitial cystitis and/or painful bladder syndrome and/or bladder pain syndrome” as used herein primarily refers to three groups of urinary symptoms, which may be defined as storage (irritative), voiding (obstructive) and post-micturition symptoms. Storage symptoms comprise urgency, frequency, nocturia, urgency incontinence and stress incontinence. Voiding symptoms comprise hesitancy, poor flow, intermittency, straining and dysuria. Post-micturition symptoms comprise terminal dribbling, post-void dribbling and a sense of incomplete emptying. The term ‘urgency’ is defined by the International Continence Society as the complaint of a sudden compelling desire to pass urine which is difficult to defer. This may be associated with a concern or fear of incontinence, a concern or fear of worsening pain, pressure or discomfort, or a concern or fear of onset or worsening of another unpleasant symptom related to the lower urinary tract. In some patients with interstitial cystitis/painful bladder syndrome/bladder pain syndrome, this sensation of urgency may be accompanied by an increasing feeling of malaise and/or nausea.

Those “in need of treatment” include mammals, such as humans, already having interstitial cystitis including those in which the disease or disorder is to be prevented.

### TNF Inhibitors

TNFα inhibitor which is used in the methods and compositions of the invention includes any agent which interferes with TNFα activity. In a preferred embodiment, the TNFα inhibitor can neutralize TNFα activity, particularly detrimental TNFα activity which is associated with interstitial cystitis, and related complications and symptoms.

In one embodiment, the TNFα inhibitor used in the invention is a TNFα antibody, or an antigen-binding fragment thereof, including chimeric, humanized, and human antibodies. Examples of TNFα antibodies which may be used in the invention include, but not limited to, infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein. Other examples of TNFα inhibitors which may be used in the methods and compositions of the invention include etanercept (Enbrel, described in WO 91/03553 and WO 09/406,476), soluble TNF receptor Type I, a pegylated soluble TNF receptor Type I (PEGs TNF-R1), p55TNFR1 gG (Lenercept), and recombinant TNF binding protein (r-TBP-I) (Serono).

In one embodiment, the invention features uses and composition for treating or determining the efficacy of a TNFα inhibitor for the treatment of interstitial cystitis, wherein the TNFα antibody is an isolated human antibody, or antigen-binding portion thereof, that binds to human TNFα with high affinity and a low off rate, and also has a high neutralizing capacity. Preferably, the human antibodies used in the invention are recombinant, neutralizing human anti-hTNFα antibodies. The most preferred recombinant, neutralizing antibody of the invention is referred to herein as D2E7, also referred to as HUMIRA® or adalimumab. The properties of D2E7 (adalimumab/HUMIRA®) have been described in Salfeld et al., U.S. Pat. Nos. 6,090,382, 6,258,562, and 6,509,015, which are each incorporated by reference herein. The methods of the invention may also be performed using chimeric and humanized murine anti-hTNFα antibodies which have undergone clinical testing for treatment of rheumatoid arthritis (see e.g., Elliott, M. J., et al. (1994) Lancet 344:1125-1127; Elliot, M. J., et al. (1994) Lancet 344:1105-1110; Rankin, E. C., et al. (1995) Br. J. Rheumatol. 34:334-342).

In one embodiment, the method of the invention includes determining the efficacy of D2E7 antibodies and antibody portions, D2E7-related antibodies and antibody portions, or other human antibodies and antibody portions with equivalent properties to D2E7, such as high affinity binding to hTNFα with low dissociation kinetics and high neutralizing capacity, for the treatment of interstitial cystitis. In one embodiment, the invention provides treatment with an isolated human antibody, or an antigen-binding portion thereof, that dissociates from human TNFα with a K_{d} of 1×10⁻⁸M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. More preferably, the isolated human antibody, or antigen-binding portion thereof, dissociates from human TNFα with a K_{off} of 5×10⁻⁴ s⁻¹ or less, or even more preferably, with a K_{off} of 1×10⁻⁴ s⁻¹ or less. More preferably, the isolated human antibody, or antigen-binding portion thereof, neutralizes human TNFα cytotoxicity in a standard in vitro L929 assay with an IC₅₀ of 1×10⁻⁸ M or less, even more preferably with an IC₅₀ of 1×10⁻⁹ M or less and still more preferably with an IC₅₀ of 1×10⁻¹⁰ M or less. In a preferred embodiment, the antibody is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the invention pertains to treating interstitial cystitis by administering human antibodies that have slow dissociation kinetics for association with hTNFα and that have light and heavy chain CDR3 domains that structurally are identical to or related to those of D2E7. Position 9 of the D2E7 VL CDR3 can be occupied by Ala or Thr without substantially affecting the K_{off}. Accordingly, a consensus motif for the D2E7 VL CDR3 comprises the amino acid sequence: Q-R—Y—N—R-A-P-Y-(T/A) (SEQ ID NO: 1). Additionally, position 12 of the D2E7 VH CDR3 can be occupied by Tyr or Asn, without substantially affecting the K_{off}. Accordingly, a consensus motif for the D2E7 VH CDR3 comprises the amino acid sequence: V-S-Y-L-S-T-A-S-S-L-D-(Y/N) (SEQ ID NO: 2). Moreover, as demonstrated in Example 2 of U.S. Pat. No. 6,090,382, the CDR3 domain of the D2E7 heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the K_{off}. Still further, the skilled artisan will appreciate that, given the amenability of the D2E7 VL and VH CDR3 domains to substitutions by alanine, substitution of other amino acids within the CDR3 domains may be possible while still retaining the low off rate constant of the antibody, in particular substitutions with conservative amino acids. Preferably, no more than one to five conservative amino acid substitutions are made within the D2E7 VL and/or VH CDR3 domains. More preferably, no more than one to three conservative amino acid substitutions are made within the D2E7 VL and/or VH CDR3 domains. Additionally, conservative amino acid substitutions should not be made at amino acid positions critical for binding to hTNFα. Positions 2 and 5 of the D2E7 VL CDR3 and positions 1 and 7 of the D2E7 VH CDR3 appear to be critical for interaction with hTNFα and thus, conservative amino acid substitutions preferably are not made at these positions (although an alanine substitution at position 5 of the D2E7 VL CDR3 is acceptable, as described above) (see U.S. Pat. No. 6,090,382).

The TNFα antibody used in the methods and compositions of the invention may be modified for improved treatment of interstitial cystitis. In some embodiments, the TNFα antibody or antigen binding fragments thereof, is chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl-ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat interstitial cystitis by administration of the TNFα antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In yet another embodiment of the invention, TNFα antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity.

An antibody or antibody portion used in the methods of the invention can be derivatized or linked to another functional molecule (e.g., another peptide or protein). Accordingly, the antibodies and antibody portions of the invention are intended to include derivatized and otherwise modified forms of the human anti-hTNFα antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Uses and Compositions for Treating Interstitial Cystitis

In one embodiment of the invention, a TNFα inhibitor is used to treat a pain and/or a lower urinary tract symptom(s) (LUTS) associated with interstitial cystitis and/or painful bladder syndrome and/or bladder pain syndrome.

Methods of treatment described herein may include administration of a TNFα inhibitor to a subject to achieve a therapeutic goal, e.g., treatment of a pain and/or a lower urinary tract symptom(s) (LUTS) associated with interstitial cystitis and/or painful bladder syndrome and/or bladder pain syndrome. Also included in the scope of the invention are uses of a TNFα inhibitor in the manufacture of a medicament to achieve a therapeutic goal, e.g., a pain and/or a lower urinary tract symptom(s) (LUTS) associated with interstitial cystitis and/or painful bladder syndrome and/or bladder pain syndrome. Thus, where methods are described herein, it is also intended to be part of this invention that the use of the TNFα inhibitor in the manufacture of a medicament for the purpose of the method is also considered within the scope of the invention. Likewise, where a use of a TNFα inhibitor in the manufacture of a medicament for the purpose of achieving a therapeutic goal is described, methods of treatment resulting in the therapeutic goal are also intended to be part of the invention.

In one embodiment, treatment of interstitial cystitis is achieved by administering a human TNFα antibody, or an antigen-binding portion thereof, to a subject having interstitial cystitis., wherein the human TNFα antibody, or an antigen-binding portion thereof, is administered on a biweekly dosing regimen. Biweekly dosing regimens can be used to treat disorders in which TNFα activity is detrimental, and are further described in U.S. application Ser. No. 10/163,657 (U.S. 20030235585), incorporated by reference herein. In one embodiment, biweekly dosing includes a dosing regimen wherein doses of a TNFα inhibitor are administered to a subject every other week beginning at week 1, week 2, week 3 or week 4. In one embodiment, biweekly dosing includes a dosing regimen where doses of a TNFα inhibitor are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing is preferably administered parenterally, including subcutaneously. In another embodiment the dosing regimen is every ten days. In one embodiment, the human TNFα antibody, or an antigen-binding portion thereof, is administered in a dose of about 40 mg. In one embodiment, the human TNFα antibody, or an antigen-binding portion thereof, is adalimumab. The invention also provides a method of treating interstitial cystitis-related disorders, comprising administering a TNFα inhibitor to a subject. The TNFα inhibitors used in the present invention may be administered by a variety of methods known in the art, although for many therapeutic applications, the preferred route/mode of administration is parenteral, including intravenous or subcutaneous injection.

In one embodiment, treatment of interstitial cystitis. is achieved using multiple variable dosing methods of treatment. Examples of such multiple variable dosing regimens are described in PCT appln. no. PCT/US05/12007, incorporated by reference herein. For example, a loading dose of about 160 mg of a TNFα inhibitor may first be administered to a subject having interstitial cystitis, followed by a second loading does or 80 mg and maintenance or treatment dose of about 40 mg. Alternatively a loading dose of about 80 mg of a TNFα inhibitor may first be administered to a subject having interstitial cystitis, and maintenance or treatment dose of about 40 mg.

In one embodiment, the invention provides a method of treating interstitial cystitis in a subject comprising administering an initial loading dose of a TNFα inhibitor to the subject at week 0. In one embodiment, the initial dose is given in its entirety on one day or is divided over 2 days. In one embodiment, the initial dose is administered subcutaneously. Following administration of the initial loading dose, a second dose, i.e., loading dose, of the TNFα inhibitor may be administered to the subject, wherein the second dose is about half the dose amount of the initial loading dose. Following administration of the second loading dose, a third dose, i.e., maintenance or treatment dose, of the TNFα inhibitor may be administered to the subject, wherein the second dose is about half the dose amount of the second loading dose. Alternatively, following administration of the initial loading dose, a second dose, i.e., maintenance or treatment dose, of the TNFα inhibitor may be administered to the subject, wherein the second dose is about half the dose amount of the initial loading dose. In one embodiment, the second dose is administered to the subject about one week after the first dose. In another embodiment the second dose is administered about two weeks after the first dose. In one embodiment the third dose is administered about 2 weeks after the second does. In one embodiment, the second dose is administered subcutaneously. Subsequent doses may be administered following the second or third dose in order to achieve treatment of the subject.

In another embodiment, the initial dose of the human TNFα antibody, or antigen-binding portion thereof, comprises 80 mg and may be given at week 0, followed by at least one maintenance dose of the human TNFα antibody, or antigen-binding portion thereof, comprising 40 mg, administered on a biweekly dosing regimen or a ten day dosing regimen.

In yet another embodiment, the initial dose of the human TNFα antibody, or antigen-binding portion thereof, comprises 160 mg and may be given at week 0, followed by at least one maintenance dose of the human TNFα antibody, or antigen-binding portion thereof, comprising 40 mg, administered on a biweekly dosing regimen or a ten day dosing regimen.

In a further embodiment, the initial dose of the human TNFα antibody, or antigen-binding portion thereof, comprises 160 mg and may be given at week 0, followed by at least a second loading dose of 80 mg, followed by at least one maintenance dose of the human TNFα antibody, or antigen-binding portion thereof, comprising 40 mg, administered on a biweekly dosing regimen or a ten day dosing regimen.

Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Dosage regimens described herein may be adjusted to provide the optimum desired response, e.g., maintaining remission of interstitial cystitis, in consideration of the teachings herein. It is to be noted that dosage values may vary with the type and severity of interstitial cystitis. It is to be further understood that for any particular subject, specific dosage regimens may be adjusted over time according to the teachings of the specification and the individual need and the professional judgment of the person administering or supervising the administration of the compositions, and that dosage amounts and ranges set forth herein are exemplary only and are not intended to limit the scope or practice of the claimed invention.

### Additional Therapeutic Agents

TNFα inhibitors, including TNFα antibodies, or antigen binding portions thereof, may be used in the methods, uses, and compositions of the invention either alone or in combination with an additional therapeutic agent. It should be understood that the TNFα inhibitors can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the TNFα inhibitors. The additional agent also can be an agent that imparts a beneficial attribute to the therapeutic composition, e.g., an agent which effects the viscosity of the composition.

It should further be understood that the combinations which are to be included within this invention are those combinations useful for their intended purpose. The agents set forth below are illustrative for purposes and not intended to be limited. The combinations, which are part of this invention, can be the TNFα inhibitors of the present invention and at least one additional agent selected from DMSO or pentosan polysulfate sodium (Elmiron®) The combination can also include more than one additional agent, e.g., two or three additional agents if the combination is such that the formed composition can perform its intended function.

### Pharmaceutical Compositions

Antibodies, antibody-portions, and other TNFα inhibitors for use in the methods of the invention, can be incorporated into pharmaceutical compositions suitable for administration to a subject. Typically, the pharmaceutical composition comprises an antibody, antibody portion, or other TNFα inhibitor, and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody, antibody portion, or other TNFα inhibitor.

The compositions for use in the methods and compositions of the invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies or other TNFα inhibitors. The preferred mode of administration is parenteral (e.g., intravenous, subcutaneous, intraperitoneal, intramuscular). In a preferred embodiment, the antibody or other TNFα inhibitor is administered by intravenous infusion or injection. In another preferred embodiment, the antibody or other TNFα inhibitor is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody, antibody portion, or other TNFα inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In one embodiment, the invention includes pharmaceutical compositions comprising an effective TNFα inhibitor and a pharmaceutically acceptable carrier, wherein the effective TNFα inhibitor may be used to treat interstitial cystitis.

In one embodiment, the antibody or antibody portion for use in the methods of the invention is incorporated into a pharmaceutical formulation as described in PCT/IB03/04502 and U.S. Appln. No. 20040033228, incorporated by reference herein. This formulation includes a concentration 50 mg/ml of the antibody D2E7 (adalimumab), wherein one pre-filled syringe contains 40 mg of antibody for subcutaneous injection.

The antibodies, antibody-portions, and other TNFα inhibitors of the present invention can be administered by a variety of methods known in the art, although for many therapeutic applications, the preferred route/mode of administration is parenteral, e.g., subcutaneous injection. In another embodiment, administration is via intravenous injection or infusion.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, Robinson, ed., Dekker, Inc., New York, 1978.

In one embodiment, the TNFα antibodies and inhibitors used in the invention are delivered to a subject subcutaneously. In one embodiment, the subject administers the TNFα inhibitor, including, but not limited to, TNFα antibody, or antigen-binding portion thereof, to himself/herself.

The TNFα antibodies and inhibitors used in the invention may also be administered in the form of protein crystal formulations which include a combination of protein crystals encapsulated within a polymeric carrier to form coated particles. The coated particles of the protein crystal formulation may have a spherical morphology and be microspheres of up to 500 micro meters in diameter or they may have some other morphology and be microparticulates. The enhanced concentration of protein crystals allows the antibody of the invention to be delivered subcutaneously. In one embodiment, the TNFα antibodies of the invention are delivered via a protein delivery system, wherein one or more of a protein crystal formulation or composition, is administered to a subject with a TNFα-related disorder. Compositions and methods of preparing stabilized formulations of whole antibody crystals or antibody fragment crystals are also described in WO 02/072636, which is incorporated by reference herein. In one embodiment, a formulation comprising the crystallized antibody fragments described in PCT/IB03/04502 and U.S. Appln. No. 20040033228, incorporated by reference herein, are used to treat rheumatoid arthritis using the treatment methods of the invention.

In certain embodiments, an antibody, antibody portion, or other TNFα inhibitor of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion for use in the methods of the invention is coformulated with and/or coadministered with one or more additional therapeutic agents, including compositions uses to treat interstitial cystitis. For example, an anti-hTNFα antibody or antibody portion of the invention may be coformulated and/or coadministered with one or more additional antibodies that bind other targets associated with TNFα related disorders (e.g., antibodies that bind other cytokines or that bind cell surface molecules), one or more cytokines, soluble TNFα receptor (see e.g., PCT Publication No. WO 94/06476) and/or one or more chemical agents that inhibit hTNFα production or activity (such as cyclohexane-ylidene derivatives as described in PCT Publication No. WO 93/19751) or any combination thereof. Furthermore, one or more antibodies of the invention may be used in combination with two or more of the foregoing therapeutic agents. Such combination therapies may advantageously utilize lower dosages of the administered therapeutic agents, thus avoiding possible side effects, complications or low level of response by the patient associated with the various monotherapies.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody, antibody portion, or other TNFα inhibitor may vary according to factors such as the disease state, age, sex, and weight of the individual, and the ability of the antibody, antibody portion, other TNFα inhibitor to elicit a desired response in the individual. A therapeutically effective amount is also one in which any toxic or detrimental effects of the antibody, antibody portion, or other TNFα inhibitor are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The invention also pertains to packaged pharmaceutical compositions or kits for administering the anti-TNF antibodies of the invention for the treatment of interstitial cystitis. In one embodiment of the invention, the kit comprises a TNFα inhibitor, such as an antibody and instructions for administration of the TNFα inhibitor for treatment of interstitial cystitis. The instructions may describe how, e.g., subcutaneously, and when, e.g., at week 0, week 2, week 4, etc., the different doses of TNFα inhibitor shall be administered to a subject for treatment.

Another aspect of the invention pertains to kits containing a pharmaceutical composition comprising a TNFα inhibitor, such as an antibody, and a pharmaceutically acceptable carrier and one or more pharmaceutical compositions each comprising an additional therapeutic agent useful for treating interstitial cystitis, and a pharmaceutically acceptable carrier. Alternatively, the kit comprises a single pharmaceutical composition comprising an anti-TNFα antibody, one or more drugs useful for treating interstitial cystitis, and a pharmaceutically acceptable carrier. The instructions may describe how, e.g., subcutaneously, and when, e.g., at week 0, week 2, week 4, etc., the different doses of TNFα inhibitor and/or the additional therapeutic agent shall be administered to a subject for treatment.

The kit may contain instructions for dosing of the pharmaceutical compositions for the treatment of interstitial cystitis.

The package or kit alternatively can contain the TNFα inhibitor and it can be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### REFERENCES

1. Jones, C. A. and Nyberg, L.: Epidemiology of interstitial cystitis. Urology, Suppl, 49:2,1997.
2. Held, P. J., Hanno, P. M., Wein, A. J., Pauly, M. V. and Cann, M. A.: Epidemiology of interstitial cystitis: 2. In: Interstitial Cystitis. Edited by P. H. Hanno, D. R. Staskin, R. J. Krane and A. J. Wein. Londodn:Springer-Verlag, sect. 2, chapt. 4, pp. 29-48, 1990
3. Neal, N. E.: Interstitial cystitis: Evaluation and related conditions. J Urol, 181: 2414, 2009.
4. Kushner, L. Molwin, R. M.: Efficiency of questionnaires used to screen for interstitial cystitis.
5. IC/PBS Diet Guidelines, http://www.ichelp.org/Default.aspx?tabid=247
6. DMSO: Many uses, Much Controversy, http://wwwdmso.org/articles/information/muir.htm
7. Elmiron Approved by US FDA, http://www.ic-network.com/newsroom/996.html
8. Selected Therapies For Interstitial Cystitis (Table 24-2), Campbell's Urology, 6th ed. Philadelphia, W. B. Saunders Co., 1992, pp. 991.
9. Wein, A. J.: Randomized, double blind, dose-ranging study of pentosan polysulfate sodium for interstitial cystitis. J Urol, 174: 2248, 2005.
10. Gonzales, R. R. Fong, T. Belmar, N. Saban, M. Felsen, D. Te, A.: Modulating bladder neuro-inflammation: RDP58, a novel anti-inflammatory peptide, decreases inflammation and nerve growth factor production in experimental cystitis. J Uro1173: 630, 2005.
11. Wesselmann, U: Interstitial cystitis: a chronic visceral pain syndrome. Urology, 57: 47,2001.
12. Aldenborg, F., Fall, M. and Enerback, L.: Mast cells in interstitial cystitis. Ann Urol (Paris), 23: 165, 1989.
13. Theoharides, T. C., Kempuraj, D. and Sant, G. R.: Mast cell involvement in interstitial cystitis: a review of human and experimental evidence. Urology, suppl., 57: 47, 2001.
14. Bjorling, D. E., Jerde, T. J., Zine, M. J., Busser, B. W., Saban, M. R., Saban, R.: Mast cells mediate the severity of experimental cystitis in mice. J Urol, 162: 231, 1999.
15. Sant, G. R. Theoharides, T. C.: The role of the mast cell in interstitial cystitis. Urol Clin North Am, 21: 41, 1994.
16. Hanno, P. Levin, R. M. Monson, F. C. Teuscher, C. Zhou, Z. Z. Ruggieri, M. et al: Diagnosis of interstitial cystitis. J Urol, 143: 278, 1990.
17. Theoharides, T. C. Sant, G. R. el-Mansoury, M. Letourneau, R. Ucci, A. A. Meares, E. M.: Activation of bladder mast cells in interstitial cystitis: a light and electron microscopic study. J Urol, 153: 629,1995.
18. Bingham, C. O., 3rd Austen, K. F.: Mast-cell responses in the development of asthma. J Allergy Clin Immunol, 105: 5527, 2000.
19. Marshall, J. S. Bienenstock, J.: The role of mast cells in inflammatory reactions in the airways, skin, and intestine, Curr Opin Immunol, 6: 853, 1994.
20. Thelharides, T. C. Cochrane, D. E.: Critical role of mast cells in inflammatory diseases and the effect of acute stress. J Neuroimmunol, 146: 1, 2004.
21. Batler, R. A. Sengupta, S. Forrestal, S. G. Schaeffer, A. J. Klumpp, D. J.: Mast cell activation triggers a urothelial inflammatory response mediated by tumor necrosis factor-a. J Urol, 168: 819, 2002.
22. Chen, M. C. Blunt, L. W. Pins, M. R. Klumpp, D. J.: Tumor necrosis factor promotes differential trafficking of bladder mast cells in neurogenic cystitis. J Urol, 175: 754, 2006.
23. Boucher, W. Kempuraj, D. Coa, J. Papaliodis, D. Theoharides, T. C.: Intravesical suplatast tosilate (IPD-1151T) inhibits experimental bladder inflammation. J Urol, 177: 1186, 2007.
24. Boucher, W. Stern, J. M. Kotsinyan, V. Kempuraj, D. Papaliodis, M. S. Cohen, M. S.: Intravesical nanocrystalline silver decreases experimental bladder inflammation. J Urol, 179: 1598, 2008.
25. Interstitial Cystitis Support Group, MD Junction, 37-year old veteran http://www.mdjunction.com/forums/interstitial-cystitis-discussions/introductions-personal-stories/786801-37year-ic-veteran-remicade-did-the-trick
26. Theoharides, T. C. Sant, G. R.: Immunomodulators for treatment of interstitial cystitis. Urology, 65: 633, 2005.

### EXAMPLES

### Example 1

### Case Study

A male patient who was being treated with monthy intravesical instillations of DMSO for interstitial cystitis, noticed improvement in his bladder sysmptoms after receiving Humira® 40 mg. He received a loading dose Humira® followed by a maintance does of 40 mg q every 10 days to treat his Crohn's disease. He has complained of bladder pain, urininary urgency, urinary voiding frequency and nocturia for more than 10 years. All these symptoms have resolved since receiving Humira®.

We claim:

1. A method of treating or alleviating a symptom of interstitial cystitis in a subject in need thereof comprising, administering a therapeutically effective amount of Humira®.

2. The method of claim 1, wherein said Humira® is administered subcutaneously.

3. The method of claim 1, wherein said therapeutically effective amount is 40 mg.

4. The method of claim 1, wherein said Humira® is administered in an initial loading dose followed by a maintenance dose.

5. The method of claim 4, wherein said loading dose is 160 mg.

6. The method of claim 4, wherein said maintenance dose is 40 mg.

7. The method of claim 4, wherein said maintenance dose is administered bi-weekly.

8. The method of claim 4, wherein a second loading does in administered after said initial loading dose.

9. The method of claim 8, wherein said second loading dose is 80 mg.

10. The method of claim 4, wherein said initial loading dose is administered over two consecutive days.

11. The method of claim 4, wherein said maintenance dose is administered every ten days.


SEQUENCE LISTING (2 sequences)

SEQ ID NO: 1 - protein, 9 aa, Artificial Sequence
  Chemically synthesized peptide
QRYNRAPYX

SEQ ID NO: 2 - protein, 12 aa, Artificial Sequence
  Chemically synthesized peptide
VSYLSTASSLDX